(19) United States
(12) Patent Application Publication
(10) Pub. No.: US 2004/0116327 A1
(43) Pub. Date: Jun. 17, 2004

(54) Use of ppar-alpha-gamma ligands or agonists to prevent the rupture of atherosclerotic plaques

(76) Inventors: Samuel D. Wright, Westfield, NJ (US); Tian-Quan Cai, Edison, NJ (US)

Correspondence Address: MERCK AND CO INC, P O BOX 2000, RAHWAY, NJ, 070650907

(21) Appl. No.: 10/475,036

(22) PCT Filed: Apr. 12, 2002
(86) PCT No.: PCT/US02/11991

(30) Foreign Application Priority Data
Apr. 18, 2001 (US) 60284748

(51) Int. Cl. A61K 31/00

(52) U.S. Cl. 514/1

(57) ABSTRACT

The invention encompasses a method for preventing the rupture of atherosclerotic plaques in a mammalian patient in need of such prevention comprising administering to said patient a compound that is capable of simultaneously binding or binding and activating PPARα and PPARγ or concomitantly administering a selective PPARα agent with a selective PPARγ agent in an amount that is effective to prevent the rupture of atherosclerotic plaques. The invention also encompasses a method for increasing atherosclerotic plaque stability in a mammalian patient in need thereof comprising administering to said patient a compound that is capable of simultaneously binding or binding and activating PPARα and PPARγ or concomitantly administering a selective PPARα agent with a selective PPARγ agent in an amount that is effective to increase plaque stability.

### BACKGROUND OF THE INVENTION

Peroxisome proliferator-activated receptors (PPARs) are transcription factors belonging to the nuclear receptor supergene family. Three distinct PPARs, termed α, δ and γ, have been described. Each one is encoded by a separate gene. PPARs are characterized by distinct tissue distribution patterns and metabolic functions.

Atherosclerotic plaques may physically occlude the flow of blood through arteries. However, the primary mechanism through which atherosclerotic plaques cause morbidity and mortality involves the formation of an occlusive clot in an artery with sudden and dramatic decrease of blood flow. Clots form when the thrombogenic interior of an atherosclerotic plaque is exposed to flowing blood. This occurs during rupture of the plaque.

It is appreciated that plaques with a thick fibrous cap are physically strong, resistant to rupture, and present a low health risk. In contrast, plaques with a thin or incomplete fibrous cap (vulnerable plaques) are prone to rupture and present a large health risk. It is an object of this invention to promote the stability of plaques to rupture.

The fibrous cap of atherosclerotic plaques is made up of extracellular matrix proteins such as collagen and elastin. These proteins are degraded by proteases such as matrix metalloproteinase 9 (MMP-9) secreted by stimulated macrophages within the plaque. Recent studies have shown that PPARγ agonists reduce MMP-9 expression in cultured macrohages {(Nature 391: 79, 1998; Am. J. Pathol. 153: 17, 1998)}. It is an objective of this invention to provide an improved means of suppressing MMP-9 expression. We find that the combination of PPARα and PPARγ agonists gives more suppression of MMP-9 expression than either agent individually. It is an object of this invention to decrease MMP-9 expression and increase plaque stability by administering a compound that is capable of simultaneously binding or simultaneously binding and activating PPARα and PPARγ or concomitantly administering a PPARα agent with a PPARγ agent.

### SUMMARY OF THE INVENTION

The invention encompasses a method for preventing the rupture of atherosclerotic plaques in a mammalian patient in need of such prevention comprising administering to said patient a compound that is capable of simultaneously binding or binding and activating PPARα and PPARγ or concomitantly administering a selective PPARα agent with a selective PPARγ agent in an amount that is effective to prevent the rupture of atherosclerotic plaques. The invention also encompasses a method for increasing atherosclerotic plaque stability in a mammalian patient in need thereof comprising administering to said patient a compound that is capable of simultaneously binding or binding and activating PPARα and PPARγ or concomitantly administering a selective PPARα agent with a selective PPARγ agent in an amount that is effective to increase plaque stability.

### DETAILED DESCRIPTION OF THE INVENTION

The present invention encompasses a method for preventing the rupture of atherosclerotic plaques in a mammalian patient in need of such prevention comprising administering to said patient a compound that is capable of simultaneously binding PPARα and PPARγ or concomitantly administering a compound that selectively binds PPARα with a compound that selectively binds PPARγ in an amount that is effective to prevent the rupture of atherosclerotic plaques.

Compounds that are capable of simultaneously binding PPARα and PPARγ or “dual ligands” are defined as those compounds with half-maximal concentration potencies (IC₅₀'s or KI's) for displacement of radioligand binding to hPPARγ vs. hPPARα that differ by less than 30-fold as measured by the human PPARα and PPARγ binding assays described below. All other compounds binding to PPARα and/or PPARγ that fall outside this definition are considered compounds that selectively bind either PPARα or PPARγ for purposes of this specification.

An embodiment of the invention encompasses administering the compound that is capable of simultaneously binding PPARα and PPARγ.

An embodiment of the invention encompasses the instant method wherein the compound that is capable of simultaneously binding PPARα and PPARγ has a half-maximal concentration potency (IC₅₀or KI) for displacement of radioligand binding to hPPARγ vs. hPPARα that differs by less than 20-fold as measured by the human PPARα and PPARγ binding assays. Another embodiment is wherein the compound that is capable of simultaneously binding PPARα and PPARγ has a half-maximal concentration potency (IC₅₀or KI) for displacement of radioligand binding to hPPARγ vs. hPPARα that differs by less than 10-fold as measured by the human PPARα and PPARγ binding assays. Another embodiment is wherein the compound that is capable of simultaneously binding PPARα and PPARγ has a half-maximal concentration potency (IC₅₀or KI) for displacement of radioligand binding to hPPARγ vs. hPPARα that differs by less than 5-fold as measured by the human PPARα and PPARγ binding assays. Another embodiment is wherein the compound that is capable of simultaneously binding PPARα and PPARγ has a half-maximal concentration potency (IC₅₀or KI) for displacement of radioligand binding to hPPARγ vs. hPPARα differ by less than 2-fold as measured by the human PPARα and PPARγ binding assays.

Another embodiment of the invention encompasses the instant method wherein the compound that is capable of simultaneously binding PPARα and PPARγ is orally active. For purposes of this specification, compounds that are orally active means compounds that produce a therapeutic response following oral ingestion of the compound.

Another embodiment of the invention encompasses the instant method wherein the compound that is capable of simultaneously binding PPARα and PPARγ possesses a long duration of action. For purposes of this specification, compounds that possess a long duration of action means compounds that have a half-life equal to or exceeding about 1 hour.

The compounds of the present invention that can be used to prevent the rupture of atherosclerotic plaques have both PPARα and PPARγ activity, which are defined in terms of PPAR agonism or in terms of binding to the PPAR receptors, partially displacing other compounds that are excellent PPAR ligands.

Another embodiment of the invention encompasses a method for preventing the rupture of atherosclerotic plaques in a mammalian patient in need of such prevention comprising administering to said patient a compound that is capable of simultaneously binding and activating PPARα and PPARγ or concomitantly administering a compound that selectively binds and activates PPARα with a compound that selectively binds and activates PPARγ in an amount that is effective to prevent the rupture of atherosclerotic plaques.

Compounds that are capable of simultaneously binding and activating PPARα and PPARγ or “dual PPARα/PPARγ agonists” are defined as those compounds that exhibit both significant PPARα and PPARγ agonism as well as half-maximal concentration potencies (EC₅₀'s) for activation of hPPARγ vs. hPPARα that differ by less than 30-fold as measured by the cell-based transactivation assay or cell-free co-activator association assay, which are described below. Compounds that exhibit both significant PPARα and PPARγ agonism are those compounds exhibiting ≧50% of the maximal effects of rosiglitazone (on human PPARγ) and ≧50% of the maximal effects of fenofibrate (on human PPARα) on both receptors as measured by the cell-based transactivation assay or cell-free co-activator association assay. It is these compounds that also exhibit half-maximal concentration potencies (EC₅₀'s) for activation of hPPARγ vs. hPPARα that differ by less than 30-fold that are considered “dual PPARα/PPARγ agonists” for purposes of this specification.

A compound that exhibits ≧50% of the maximal effects of rosiglitazone on human PPARγ but is outside the 30 fold activation difference described above is considered a compound that selectively binds and activates PPARγ. Rosiglitazone is an example of such a compound. Likewise, a compound that exhibits ≧50% of the maximal effects of fenofibrate on human PPARα but is outside the 30 fold activation difference described above is considered a compound that selectively binds and activates PPARα. Fenofibrate is an example of such a compound.

An embodiment of the invention encompasses administering the compound that is capable of simultaneously binding and activating PPARα and PPARγ.

Within this embodiment of the invention is encompassed the above method wherein the compound that is capable of simultaneously binding and activating PPARα and PPARγ has a half-maximal concentration potency (EC₅₀) for activation of hPPARγ vs. hPPARα that differs by less than 20-fold as measured by the cell-based transactivation assay or cell-free co-activator association assay. Also within this embodiment is encompassed the above method wherein the compound that is capable of simultaneously binding and activating PPARα and PPARγ has a half-maximal concentration potency (EC₅₀) for activation of hPPARγ vs. hPPARα that differs by less than 10-fold as measured by the cell-based transactivation assay or cell-free co-activator association assay. Also within this embodiment is encompassed the above method wherein the compound that is capable of simultaneously binding and activating PPARα and PPARγ has a half-maximal concentration potency (EC₅₀) for activation of hPPARγ vs. hPPARα that differs by less than 5-fold as measured by the cell-based transactivation assay or cell-free co-activator association assay. Also within this embodiment is encompassed the above method wherein the compound that is capable of simultaneously binding and activating PPARα and PPARγ has a half-maximal concentration potency (EC₅₀'s) for activation of hPPARγ vs. hPPARα differ by less than 2-fold as measured by the cell-based transactivation assay or cell-free co-activator association assay.

Within this embodiment of the invention is also encompassed the above method wherein the compound that is capable of simultaneously binding and activating PPARα and PPARγ is orally active. Also within this embodiment of the invention is encompassed the above method wherein the compound that is capable of simultaneously binding and activating PPARα and PPARγ possesses a long duration of action.

The invention also encompasses a method for increasing atherosclerotic plaque stability in a mammalian patient in need thereof comprising administering to said patient a compound that is capable of simultaneously binding PPARα and PPARγ or concomitantly administering a compound that selectively binds PPARα with a compound that selectively binds PPARγ in an amount that is effective to increase plaque stability. An embodiment of invention is the aforesaid method comprising administering the compound that is capable of simultaneously binding PPARα and PPARγ.

Another embodiment of the invention is a method for increasing atherosclerotic plaque stability in a mammalian patient in need thereof comprising administering to said patient a compound that is capable of simultaneously binding and activating PPARα and PPARγ or concomitantly administering a compound that selectively binds and activates PPARα with a compound that selectively binds and activates PPARγ in an amount that is effective to increase atherosclerotic plaque stability. Within this embodiment is encompassed the method comprising administering the compound that is capable of simultaneously binding and activating PPARα and PPARγ.

For purposes of this specification “concomitantly administering” means administering one compound by a route and in an amount commonly used therefor, contemporaneously or sequentially with another compound. When compounds are referred to as administered concomitantly, a pharmaceutical composition in unit dosage form containing the two drugs is preferred For purposes of this specification, a “PPARα agent” means a compound that selectively binds or binds and activates PPARα as defined above. “PPARγ agent” means a compound that selectively binds or binds and activates PPARγ.

Examples of compounds that are capable of simultaneously binding or simultaneously binding and activating PPARα and PPARγ, as well as examples of selective PPARα agents and selective PPARγ agents, are found in the following patents and published applications: WO 97/28115 published on Aug. 7, 1997; WO 00/78312 published on Dec. 28, 2000; WO 00/78313 published on Dec. 28, 2000; U.S. Pat. No. 5,847,008 granted on Dec. 8, 1998; U.S. Pat. No. 5,859,051 granted on Jan. 12, 1999; U.S. Pat. No. 6,008,237 granted on Dec. 28, 1999; U.S. Pat. No. 6,090,836 granted on Jul. 18, 2000; U.S. Pat. No. 6,090,839 granted on Jul. 18, 2000; U.S. Pat. No. 6,160,000 granted on Dec. 12, 2000; and U.S. Pat. No. 6,200,998 granted on Mar. 13, 2001, all of which are hereby incorporated by reference in their entirety.

Utilities

The present compounds are useful for preventing the rupture of and increasing the stability of atherosclerotic plaques. The most important mechanism responsible for the sudden and unpredictable onset of acute coronary syndromes is coronary plaque rupture with thrombosis. The risk of plaque rupture depends on plaque composition rather than plaque size.

Pathoanatomical studies have identified collagen degradation as one of the major determinants of plaque's vulnerability to rupture. Macrophages influence many aspects of atherosclerosis. By secreting matrix metalloproteinase 9 (MMP-9), macrophages directly influence the vulnerability of plaques. Macrophage-derived MMP-9 increases matrix breakdown in plaques, thereby predisposing them to rupture. MMP-9 production can be inhibited by dosing with PPARα and PPARγ agonists together or with PPARα/γ agents.

Pharmaceutical Compositions

The pharmaceutical compositions of the present invention comprise a compound that is capable of simultaneously binding or simultaneously binding and activating PPARα and PPARγ, or a combination of a selective PPARα agent with a selective PPARγ agent, as an active ingredient or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids.

The term “composition”, as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the present compounds can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form, in which case solid pharmaceutical carriers are obviously employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

The present compounds may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

Salts

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanol amine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that, as used herein, references to compounds capable of simultaneously binding or simultaneously binding and activating PPARα and PPARγ are meant to also include the pharmaceutically acceptable salts thereof. Likewise, references to compounds that are selective PPARα agents or selective PPARγ agents are meant to include pharmaceutically acceptable salts thereof.

Optical Isomers—Diastereomers—Geometric Isomers—Tautomers

The compounds of the present invention may contain one or more asymmetric centers and can thus occur as racemates and racemic mixtures, single enantiomers, diastereomeric mixtures and individual diastereomers. The present invention is meant to comprehend all such isomeric forms.

The compounds encompassed by the present invention may contain olefinic double bonds, and unless specified otherwise, are meant to include both E and Z geometric isomers.

The compounds encompassed by the present invention may exist with different points of attachment of hydrogen, referred to as tautomers. Such an example may be a ketone and its enol form, known as keto-enol tautomers. The individual tautomers as well as mixtures thereof are encompassed with compounds of Formula II and IIa.

The compounds encompassed by the present invention may be separated into diastereoisomeric pairs of enantiomers by, for example, fractional crystallization from a suitable solvent, for example methanol or ethyl acetate or a mixture thereof. The pair of enantiomers thus obtained may be separated into individual stereoisomers by conventional means, for example by the use of an optically active acid as a resolving agent.

Alternatively, any enantiomer of the compounds of the present invention may be obtained by stereospecific synthesis using optically pure starting materials or reagents of known configuration.

Administration and Dose Ranges

Any suitable route of administration may be employed for providing a mammal, and especially a human, with an effective dosage of the present compounds for the prevention of atherosclerotic plaques. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably the compounds are administered orally.

The effective dosage of the active ingredient employed may vary depending on the particular compound employed, the mode of administration, the condition being treated and the severity of the condition being treated. Such dosage may be ascertained readily by a person skilled in the art.

When preventing atherosclerotic plaques generally satisfactory results are obtained when the compound is administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams, preferably from about 1 milligrams to about 50 milligrams. In the case of a 70 kg adult human, the total daily dose will generally be from about 7 milligrams to about 350 milligrams. This dosage regimen may be adjusted to provide the optimal therapeutic response.

A preferred dosage range is from less than 1 mg per day to greater than 100 mg/day, with appropriate doses being administered to patients at risk of myocardial infarction or TIA.

Biological Assays

Standardized Cell-Based GAL4 Chimeric Receptor Transactivation Assay (Cell-Based Transactivation Assay)

The following assay is also described in: Berger J, Leibowitz M D, Doebber T W, Elbrecht A, Zhang B, Zhou G, Biswas C, Cullinan C A, Hayes N S, Li Y, Tanen M, Ventre J, Wu M S, Berger G D, Mosley R, Marquis R, Santini C, Sahoo S P, Tolman R L, Smith R G, Moller D E. Novel peroxisome proliferator-activated receptorγ (PPARγ) and PPARδ ligands produce distinct biological effects, 1999 J Biol Chem 274: 6718-6725, herein incorporated by reference in its entirety:

Expression constructs are prepared by inserting cDNA sequences encoding the ligand binding domains of human PPARγ or PPARα adjacent to the yeast GAL4 transcription factor DNA binding domain in the mammalian expression vector pcDNA3 to create pcDNA3-hPPARγ/GAL4 and pcDNA3-hPPARα/GAL4, respectively. The GAL4-responsive reporter construct, pUAS(5×)-tk-luc, contains 5 copies of the GAL4 response element placed adjacent to the thymidine kinase minimal promoter and the luciferase reporter gene. The transfection control vector, pCMV-lacZ, contains the galactosidase Z gene under the regulation of the cytomegalovirus promoter. COS-1 cells are seeded at 1.2×10⁴ cells/well in 96 well plates in Dulbecco's modified Eagle medium (high glucose) containing 10% charcoal stripped fetal calf serum, nonessential amino acids, 100 units/ml Penicillin G and 100 μg/ml Streptomycin sulfate at 37° C. in a humidified atmosphere of 10% CO₂. After 24 h, transfections are performed with Lipofectamine (Gibco-BRL, Gaithersburg, MD) according to the instructions of the manufacturer. Transfection mixes contain 0.00075 Tg of PPARγ/GAL4 or PPARα/GAL4 expression vector, 0.045 Tg of reporter vector pUAS(5×)-tk-luc and 0.0002 Tg of pCMV-lacZ vector as an internal control of transfection efficiency. Compounds are characterized by incubation with transfected cells for 48 h across a range of 8-12 concentrations from 0.1 nM to 50 uM. Cell lysates are prepared from washed cells using Reporter Lysis Buffer (Promega) according to the manufacturer's directions. Luciferase activity in cell extracts is determined using Luciferase Assay Buffer (Promega) in a ML3000 luminometer (Dynatech Laboratories). β-galactosidase activity is determined using β-D-galactopyranoside (Calbiochem-Novabiochem, LaJolla, Calif.) as described by Hollons and Yoshimura (Anal. Biochem, 182,411-418, 1989). Rosiglitazone can be used as a standard for human PPARγ activity. EC₅₀values for Rosiglitazone in the hPPARγ/GAL4 assay usually range from 20-40 nM. Fenofibrate can be used as a standard for hPPARα activity. EC₅₀ values for Fenofibrate in the hPPARγ/GAL4 assay usually range from 5-20 uM. Similarly, methods involving the co-transfection of full-length PPARγ or PPARγ along with a relevant reporter gene into one of several mammalian (or yeast) cell types could be employed as an alternative method to identify compounds with both PPARα and PPARγ agonist activity.

Cell-Free Co-Activator Association Assay

This assay measures the ability of compounds to promote the association of PPARγ (or its isolated ligand binding domain) or PPARα (or its isolated ligand binding domain) with a protein (or portion of a protein) that is (or is derived from) a co-activator molecule such as Creb Binding Protein (CBP) or Steroid Receptor Coactivator 1 (SRC-1) and can be used to identify compounds with both PPARα and PPARγ agonist activity. This assay is described in: Zhou G, Cummings R, Li Y, Mitra S, Wilkinson H, Elbrecht A, Hermes J D, Schaeffer J M, Smith R G, Moller D E. Nuclear receptors have distinct affinities for co-activators: characterization by fluorescence resonance energy transfer. Mol Endocrinol 1998 12:1594-1604, herein incorporated by reference in its entirety

Human PPARα and PPARγ binding assays

An alternative to measuring agonist activity of compounds in cell-based transactivation assays or cell-free co-activator association assays is to determine that compounds can function as ligands by binding to both PPARγ and PPARα. Compounds with half-maximal concentration potencies (IC₅₀'s or KI's) for displacement of radioligand binding to hPPARγ vs. hPPARα that differ by less than 30-fold and preferably less than 10-fold can be considered as dual ligands. For these assays, the methods described below can be employed (as also described in: Berger J, Leibowitz M D, Doebber T W, Elbrecht A, Zhang B, Zhou G, Biswas C, Cullinan C A, Hayes N S, Li Y, Tanen M, Ventre J, Wu M S, Berger G D, Mosley R, Marquis R, Santini C, Sahoo S P, Tolman R L, Smith R G, Moller D E. Novel peroxisome proliferator-activated receptorγ (PPARγ) and PPARδ ligands produce distinct biological effects, 1999 J Biol Chem 274: 6718-6725, herein incorporated by reference in its entirety):

Human PPARγ₂ and human PPARα were expressed as a GST-fusion protein in E. coli. The full length human cDNA for PPARγ₂ was subcloned into the pGEX-2T expression vector (Pharmacia). The full length human cDNA for PPARα was subcloned into the pGEX-KT expression vector (Pharmacia). E. coli containing the respective plasmids were propagated, induced, and harvested by centrifugation. The resuspended pellet was broken in a French press and debris was removed by centrifugation at 12,000×g. Recombinant human PPAR receptors were purified by affinity chromatography on glutathione sepharose. After application to the column, and one wash, receptor was eluted with glutathione. Glycerol (10%) was added to stabilize the receptor and aliquots were stored at −80° C.

For each assay, an aliquot of receptor was incubated in TEGM (10 mM Tris, pH 7.2, 1 mM EDTA, 10% glycerol, 7 μL/100 ml β-mercaptoethanol, 10 mM Na molybdate, 1 mM dithiothreitol, 5 μg/mL aprotinin, 2 μg/mL leupeptin, 2 μg/mL benzamidine and 0.5 mM PMSF) containing 0.1% non-fat dry milk and 10 nM [³H₂]L-746,962, (21 Ci/mmole), ±test compound. Assays were incubated for ˜16 hr at 4° C. in a final volume of 150 μL. Unbound ligand was removed by incubation with 100 μL dextran/gelatin-coated charcoal, on ice, for 10 min. After centrifugation at 3000 rpm for 10 min at 4° C., 50 μL of the supernatant fraction was counted in a Topcount. In this assay the KD for L-746,962 is ≈1 nM.

For a human PPARα binding assay, an aliquot of receptor was incubated in TEGM (10 mM Tris, pH 7.2, 1 mM EDTA, 10% glycerol, 7 μL/100 ml β-mercaptoethanol, 10 Na molybdate, 1 mM dithiothreitol, 5 μ/mL aprotinin, 2 μg/mL leupeptin, 2 μg/mL benzamide and 0.5 mM PMSF) containing 0.1% non-fat dry milk and 5.0 nM [³H₂]L-783483, ±test compound. Assays were incubated for ˜16 hr at 4° C. in a final volume of 150 μL. Unbound ligand was removed by incubation with 100 μL dextran/gelatin-coated charcoal, on ice, for ˜10 min. After centrifugation at 3000 rpm for 10 min at 4° C., 50 μL of the supernatant fraction was counted in a Topcount.

Cell Proliferation Assay

This assay measures the ability of cells to convert MTS tetrazolium into formazan, using the AQᵤₑₒᵤₛ cell proliferation assay kit (Promega, Madison, Wis.). This conversion is presumably accomplished by NADPH or NADH produced by dehydrogenase enzymes in metabolically active cells. The assay is described in Shu, et al., Biochemical and Biophysical Research Communications, vol. 267, pp. 345-349 (2000).

MMP-9 ELISA

This assay is used for measuring the amount of MMP-9 secreted from cultured human monocytic THP-1 in response to lipopollysaccharide (LPS) stimulation. The assay is described in Shu, et al., Biochemical and Biophysical Research Communications, vol. 267, pp. 345-349 (2000). Cultured THP-1 cells were treated with PPARα and/or γ agonists for 2 hr at 37° C. The cells were then stimulated with bacteria LPS (1 ng/ml). After 48 hr incubation at 37° C., culture supernatants were collected. MMP-9 secreted in the media were measured by ELISA using antibodies specific to MMP-9.

Dosing with PPARα and PPARγ agents together or with a dual PPARα/γ agent produces an unexpectedly superior decrease in the secretion of MMP-9 as compared to the either a PPARα or PPARγ agent alone.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A method for preventing the rupture of atherosclerotic plaques in a mammalian patient in need of such prevention comprising administering to said patient a compound that is capable of simultaneously binding PPARα and PPARγ or concomitantly administering a compound that selectively binds PPARα with a compound that selectively binds PPARγ in an amount that is effective to prevent the rupture of atherosclerotic plaques.

2. The method according to claim 1 comprising administering the compound that is capable of simultaneously binding PPARα and PPARγ.

3. The method according to claim 2 wherein the compound that is capable of simultaneously binding PPARα and PPARγ has a half-maximal concentration potency (IC₅₀or KI) for the displacement of radioligand binding to hPPARγ vs. hPPARα that differs by less than 20-fold as measured by the human PPARα and PPARγ binding assays.

4. The method according to claim 2 wherein the compound that is capable of simultaneously binding PPARα and PPARγ has a half-maximal concentration potency (IC₅₀or KI) for the displacement of radioligand binding to hPPARγ vs. hPPARα that differs by less than 10-fold as measured by the human PPARα and PPARγ binding assays.

5. The method according to claim 2 wherein the compound that is capable of simultaneously binding PPARα and PPARγ has a half-maximal concentration potency (IC₅₀or KI) for the displacement of radioligand binding to hPPARγ vs. hPPARα that differs by less than 5-fold as measured by the human PPARα and PPARγ binding assays.

6. The method according to claim 2 wherein the compound has a half-maximal concentration potency (IC₅₀or KI) for the displacement of radioligand binding to hPPARγ vs. hPPARα that differs by less than 2-fold as measured by the human PPARα and PPARγ binding assays.

7. The method according to claim 2 wherein the compound that is capable of simultaneously binding PPARα and PPARγ is orally active.

8. The method according to claim 2 wherein the compound that is capable of simultaneously binding PPARα and PPARγ possesses a long duration of action.

9. A method for preventing the rupture of atherosclerotic plaques in a mammalian patient in need of such prevention comprising administering to said patient a compound that is capable of simultaneously binding and activating PPARα and PPARγ or concomitantly administering a compound that selectively binds and activates PPARα with a compound that selectively binds and activates PPARγ in an amount that is effective to prevent the rupture of the artherosclerotic plaques in accordance with claim 1.

10. The method according to claim 9 comprising administering the compound that is capable of simultaneously binding and activating PPARα and PPARγ.

11. The method according to claim 10 wherein the compound that is capable of simultaneously binding and activating PPARα and PPARγ has a half-maximal concentration potency (EC₅₀) for activation of hPPARγ vs. hPPARα that differs by less than 20-fold as measured by the cell-based transactivation assay or cell-free co-activator association assay.

12. The method according to claim 10 wherein the compound that is capable of simultaneously binding and activating PPARα and PPARγ has a half-maximal concentration potency (EC₅₀) for activation of hPPARγ vs. hPPARα that differs by less than 10-fold as measured by the cell-based transactivation assay or cell-free co-activator association assay.

13. The method according to claim 10 wherein the compound that is capable of simultaneously binding and activating PPARα and PPARγ has a half-maximal concentration potency (EC₅₀) for activation of hPPARγ vs. hPPARα that differs by less than 5-fold as measured by the cell-based transactivation assay or cell-free co-activator association assay.

14. The method according to claim 10 wherein the compound that is capable of simultaneously binding and activating PPARα and PPARγ has a half-maximal concentration potency (EC₅₀) for activation of hPPARγ vs. hPPARα that differs by less than 2-fold as measured by the cell-based transactivation assay or cell-free co-activator association assay.

15. The method according to claim 10 wherein the compound that is capable of simultaneously binding and activating PPARα and PPARγ is orally active.

16. The method according to claim 10 wherein the compound that is capable of simultaneously binding and activating PPARα and PPARγ possesses a long duration of action.

17. A method for increasing atherosclerotic plaque stability in a mammalian patient in need thereof comprising administering to said patient a compound that is capable of simultaneously binding PPARα and PPARγ or concomitantly administering a compound that selectively binds PPARα with a compound that selectively binds PPARγ in an amount that is effective to increase plaque stability.

18. The method according to claim 17 comprising administering the compound that is capable of simultaneously binding PPARα and PPARγ.

19. A method for increasing atherosclerotic plaque stability in a mammalian patient in need thereof comprising administering to said patient a compound that is capable of simultaneously binding and activating PPARα and PPARγ or concomitantly administering a compound that selectively binds and activates PPARα with a compound that selectively binds and activates PPARγ in an amount that is effective to increase atherosclerotic plaque stability in accordance with claim 17.

20. The method according to claim 19 comprising administering the compound that is capable of simultaneously binding and activating PPARα and PPARγ.
